(19) United States
(12) Patent Application Publication
(10) Pub. No.: US 2025/0058030 A1
(43) Pub. Date: Feb. 20, 2025

(54) ULTRASONIC BARRIER CHAMBER FOR FLUID SYSTEMS

(71) Applicant: Fresenius Medical Care Holdings, Inc., Waltham, MA (US)

(72) Inventors: Martin Crnkovich, Walnut Creek, CA (US); David Yuds, Hudson, NH (US)

(21) Appl. No.: 18/234,026

(22) Filed: Aug. 15, 2023

(51) Int. Cl. A61M 1/16; A61M 1/34; A61M 1/36; G01N 29/032

(52) U.S. Cl. CPC A61M 1/1603; A61M 1/3482; A61M 1/36224; A61M 1/3627; G01N 29/032; A61M 2205/33

(57) ABSTRACT

Disclosed herein is an ultrasonic barrier chamber for fluid systems, for example, which facilitates making fluid lines airless using ultrasonic signals. In some examples, the fluid lines may include an extracorporeal blood circuit, for instance, for use in a dialysis process. In one example, a device for monitoring an extracorporeal circuit in which a blood pump is disposed to convey blood may include one or more ultrasonic transducers positioned in a predetermined vicinity of a vent of a drip chamber of the extracorporeal circuit, and a controller coupled to the one or more ultrasonic transducers, the controller configured to cause the one or more ultrasonic transducers to emit ultrasonic signals that displace particles in contents of the drip chamber away from the vent. Other embodiments are described.

### FIELD OF DISCLOSURE

The present disclosure generally relates to dialysis machines and systems, particularly to a dialysis (e.g., hemodialysis) system including a detector assembly and an ultrasonic barrier chamber that facilitates making dialysis bloodlines airless using ultrasonic signals.

### BACKGROUND

Dialysis machines are known for use in the treatment of renal disease. The two principal dialysis methods are hemodialysis (HD) and peritoneal dialysis (PD). An HD system is arranged and configured to, inter alia, pump a patient's blood through a dialyzer. In addition, the HD system includes an HD machine and one or more containers containing a fluid (e.g., a dialysate), which during use, is also pumped through the dialyzer of the HD system simultaneously with the patient's blood. A semi-permeable membrane in the dialyzer separates the blood from the dialysate within the dialyzer, allowing diffusion and osmosis exchanges between the dialysate and the blood. Thus arranged, in use, the patient's blood is cleaned or filtered.

During PD, a patient's peritoneal cavity is periodically infused with dialysis solution or dialysate. The membranous lining of the patient's peritoneum acts as a natural semi-permeable membrane that allows diffusion and osmosis exchanges between the solution and the bloodstream. These exchanges across the patient's peritoneum, like the continuous exchange across the dialyzer in HD, result in the removal of waste products, including solutes like urea and creatinine, from the blood and regulate the levels of other substances, such as sodium and water, in the blood.

During an extracorporeal fluid treatment, blood is circulated through an extracorporeal fluid circuit to filter waste from the blood. A pump can be operated to generate a flow of blood drawn from the patient through an arterial line set, through a filter, then back into the patient through a venous line set. The venous line set, and the arterial line set can be connected to the patient to enable the blood to be drawn from the patient and to be returned to the patient after the blood flows through the filter. At the end of the extracorporeal treatment, some residual blood that has not been returned to the patient during the extracorporeal treatment may remain with the arterial line set and the venous line set. In some cases, a syringe can be connected to the arterial line set or the venous line set and can be manually actuated to exert positive pressure on the residual blood within the arterial line set and the venous line set to return the blood to the patient.

### SUMMARY

This Summary is provided to introduce a selection of concepts in a simplified form that is further described below in the Detailed Description. This Summary is not intended to necessarily identify key features or essential features of the claimed subject matter, nor is it intended as an aid in determining the scope of the claimed subject matter.

In one embodiment, a device for monitoring an extracorporeal circuit in which a blood pump is disposed to convey blood may include one or more ultrasonic transducers positioned in a predetermined vicinity of a vent of a drip chamber of the extracorporeal circuit; and a controller coupled to the one or more ultrasonic transducers, the controller configured to cause the one or more ultrasonic transducers to emit ultrasonic signals that displace particles in contents of the drip chamber away from the vent.

In some embodiments of the device, the controller causes the one or more ultrasonic transducers to emit the ultrasonic signals of a predetermined frequency, the predetermined frequency is configured to displace the particles larger than a predetermined size.

In various embodiments of the device, the particles that are larger than the predetermined size cause condensation on the vent.

In some embodiments of the device, the particles that are displaced by the one or more ultrasonic transducers include one or more of water, plasma, or red blood cells.

In various embodiments of the device, the ultrasonic signals of the predetermined frequency allow the particles that are smaller than the predetermined size to pass through the vent.

In some embodiments of the device, the device further includes one or more sensors to monitor a pore size of the drip chamber.

In exemplary embodiments of the device, the one or more sensors include a second set of ultrasonic transducers, the controller is coupled with the second set of ultrasonic transducers, and the controller is configured to cause the second set of ultrasonic transducers to emit ultrasonic signals of a second predetermined frequency.

In various embodiments of the device, in response to the pore size being detected to be less than a predetermined threshold, the controller performs at least one from a group of operations, the group of operations including stopping the blood pump, closing a venous clamp, or alerting a user.

In some embodiments of the device, the one or more ultrasonic transducers is a first set of ultrasonic transducers, the predetermined frequency is a first predetermined frequency, and the device comprises a second set of ultrasonic transducers that are coupled with the controller, which causes the second set of ultrasonic transducers to emit ultrasonic signals of a second predetermined frequency, different from the first predetermined frequency, the ultrasonic signals of the second predetermined frequency prevent blood clots in the contents of the drip chamber.

In various embodiments of the device, the second set of ultrasonic transducers emits ultrasonic signals continuously that prevents red blood cells from clotting.

In exemplary embodiments of the device, the second set of ultrasonic transducers emits ultrasonic signals of a third predetermined frequency, different from the first and second predetermined frequencies, the ultrasonic signals of the third predetermined frequency sever chains of red blood cells.

In one embodiment, a dialysis system may include a venous drip chamber having a vent; one or more ultrasonic transducers positioned in a predetermined vicinity of the vent; and a controller coupled with the one or more ultrasonic transducers, the controller configured to cause the one or more ultrasonic transducers to emit ultrasonic signals that displace particles in contents of the venous drip chamber away from the vent.

In some embodiments of the system, the venous drip chamber is top-vented.

In various embodiments of the system, the controller causes the one or more ultrasonic transducers to emit the ultrasonic signals of a predetermined frequency, the predetermined frequency selected to displace the particles larger than a predetermined size.

In some embodiments of the system, the system further includes one or more sensors to monitor a pore size of the drip chamber.

In exemplary embodiments of the system, in response to the pore size being detected to be less than a predetermined threshold, the controller performs at least one from a group of operations, the group of operations comprises, stopping a blood pump, closing a venous clamp, or alerting a user.

In various embodiments of the system, the one or more sensors comprise a second set of ultrasonic transducers, wherein the controller is coupled with the second set of ultrasonic transducers, the controller configured to cause the second set of ultrasonic transducers to emit ultrasonic signals of a second predetermined frequency.

In some embodiments of the system, the one or more ultrasonic transducers is a first set of ultrasonic transducers, the predetermined frequency is a first predetermined frequency, and the dialysis system includes a second set of ultrasonic transducers that are coupled with the controller, which causes the second set of ultrasonic transducers to emit ultrasonic signals of a second predetermined frequency, different from the first predetermined frequency, the ultrasonic signals of the second predetermined frequency prevent blood clots in the contents of the venous drip chamber.

In various embodiments of the system, the controller causes the second set of ultrasonic transducers to perform at least one of: emit ultrasonic signals continuously to prevent red blood cells from clotting, or emit ultrasonic signals of a third predetermined frequency, different from the first and second predetermined frequencies, the ultrasonic signals of the third predetermined frequency sever chains of red blood cells.

In one embodiment, a computer-implemented method may include detecting, using one or more sensors, a pore size of a drip chamber coupled to a pump that causes a fluid to flow through the drip chamber; and in response to the pore size of the drip chamber being below a predetermined size, causing the pump to stop the flow of fluid.

### BRIEF DESCRIPTION OF THE SEVERAL VIEWS OF THE DRAWINGS

By way of example, illustrative embodiments of the disclosed systems, devices, and methods will now be described, with reference to the accompanying drawings. To easily identify the discussion of any particular element or act, the most significant digit or digits in a reference number refer to the figure number in which that element is first introduced.

FIG. 1 illustrates an aspect of the subject matter in accordance with one embodiment.

FIG. 2 illustrates an aspect of the subject matter in accordance with one embodiment.

FIG. 3 illustrates an aspect of the subject matter in accordance with one embodiment.

FIG. 4 illustrates an aspect of the subject matter in accordance with one embodiment.

FIG. 5 illustrates an aspect of the subject matter in accordance with one embodiment.

FIG. 6 illustrates a method in accordance with one embodiment.

### DETAILED DESCRIPTION

The present embodiments will now be described more fully hereinafter with reference to the accompanying drawings, in which several exemplary embodiments are shown. However, the subject matter of the present disclosure may be embodied in many different forms and types of devices and systems for dialysis and other potential medical devices and treatments, and should not be construed as limited to the embodiments set forth herein. Rather, these embodiments are provided so that this disclosure will be thorough and complete and willfully convey the scope of the subject matter to those skilled in the art. In the drawings, like numbers refer to like elements throughout.

As detailed elsewhere herein, dialysis bloodlines for an extracorporeal circuit utilize a venous drip chamber to ensure blood is gently returned to the patient without sudden pressure or pump speed changes affecting the patient's fistula. However, this requires air in the venous drip chamber for a separation between the force of the arterial pump and the venous portion of the extracorporeal circuit. Air can potentially enter the dialysis system at various points like tubing connections or where pressure is monitored. The air causes blood clotting over time, so anti-clotting compounds like heparin are infused in the bloodline, or Citrasate (or the like) is infused in the dialysate to minimize clotting. However, neither option works for every patient, which can cause a technical challenge.

It should be noted that while the embodiments herein are described in the context of an extracorporeal circuit, the technical solutions provided herein address technical challenges in other fields where technical challenge exists to filter larger molecules from smaller molecules, particularly when such molecules are suspended in a fluid. For example, the technical solutions provided herein of using ultrasonic signals to filter, stir, or affect one or more particles and molecules in a liquid can be applied to technical challenges in oil filtration, industrial cleaning, refineries, vehicle fuel systems, etc. Features of technical solutions described herein are not to be limited to extracorporeal circuits or any other specific application unless explicitly stated.

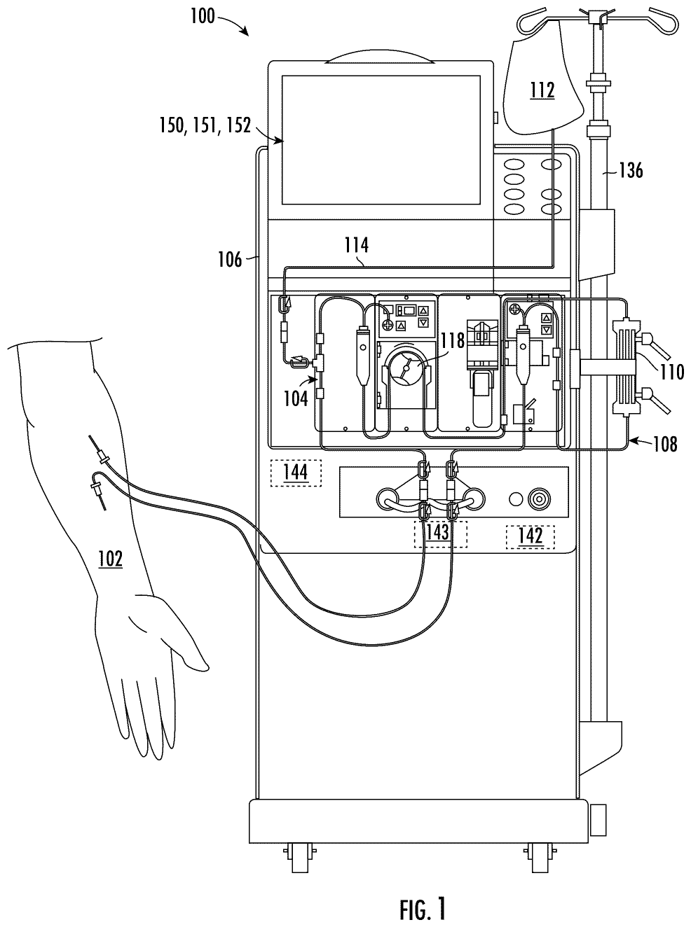

Referring to FIG. 1, during an extracorporeal treatment (e.g., a dialysis treatment) of a patient 102, an extracorporeal medical fluid treatment system 100 is operated to filter blood drawn from the patient 102. Blood from the patient 102 is drawn from the circulatory system of the patient 102 and circulated through the extracorporeal system 100. As described herein, after the extracorporeal treatment is complete, a negative pressure is generated within an arterial line set 104 to return blood to the patient 102 during a blood reinfusion process.

The extracorporeal system 100 includes an extracorporeal blood treatment machine 106, the arterial line set 104, a venous line set 108, a dialyzer 110, a fluid source 112, and a fluid line 114. In some embodiments, the machine 106 is a dialysis machine, and the extracorporeal treatment performed by the machine 106 is a dialysis treatment. The machine 106 is a reusable apparatus operated to draw blood from the patient 102 through the arterial line set 104, the dialyzer 110, the venous line set 108, and back to the patient 102. The machine 106 can be used for multiple extracorporeal treatments. The machine 106 includes a blood pump 118 operable to draw blood from the patient 102.

The arterial line set 104, the venous line set 108, and the dialyzer 110 are disposable portions of the extracorporeal system 100. Because these disposable portions of the extracorporeal system 100 come into contact with the blood, these portions can be single-use components that are discarded after a single extracorporeal treatment. After the end of the extracorporeal treatment, the arterial line set 104, the venous line set 108, and the dialyzer 110 may contain residual blood drawn from the patient 102 during the extracorporeal treatment. This blood, through the blood reinfusion process described herein, can be returned to the patient 102 to reduce blood loss.

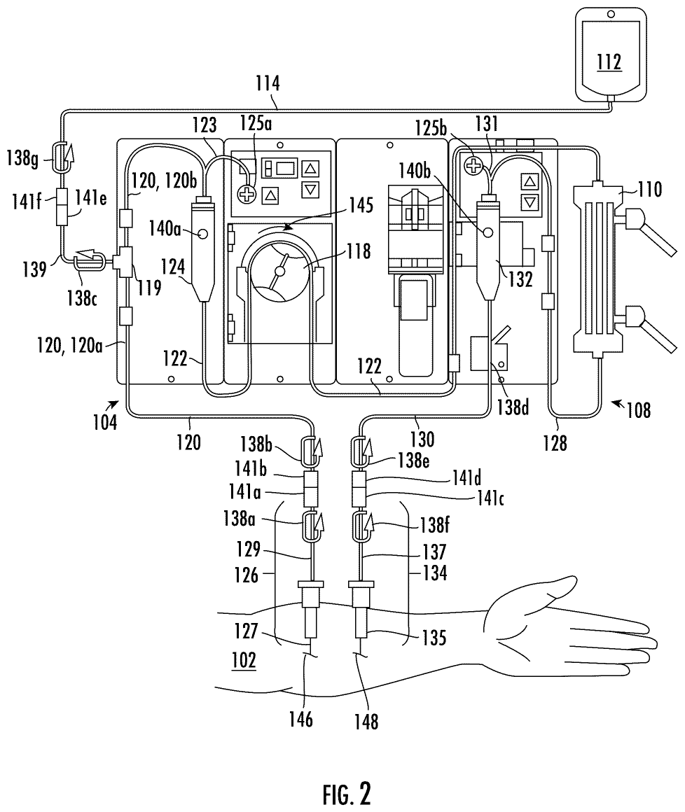

Referring to FIG. 2, the arterial line set 104 includes an arterial access line 120, an arterial line 122, and an arterial drip chamber 124. One end of the arterial access line 120 is connected to the top of the arterial drip chamber 124, and the other end of the arterial access line 120 is connectable to an arterial needle assembly 126. The arterial access line 120 includes a first portion 120a, and a second portion 120b, each connected to a fluid adapter 119 of the arterial line set 104.

The arterial line 122 extends from the bottom of the arterial drip chamber 124 to the dialyzer 110. The arterial drip chamber 124 is positioned along the arterial access line 120. A pressure transducer 125a is connected to the arterial drip chamber 124 via a pigtail line 123 extending from the arterial drip chamber 124 and is configured to detect fluid pressure within the arterial drip chamber 124.

The arterial needle assembly 126 includes a needle 127 that is insertable into the patient 102 and a fluid line 129 that is connectable to the arterial access line 120. A manually operable connector 141a at the end of the fluid line 129 is configured to be connected to a manually operable connector 141b at the end of the first portion 120 of the arterial access line 120. Like the arterial line set 104, the arterial needle assembly 126 can be a single-use disposable component that is discarded after the end of the extracorporeal treatment. During an extracorporeal treatment, the arterial needle assembly 126 is connected to the arterial access line 120 using the connectors 141a, 141b. The needle 127 of the arterial needle assembly 126 is inserted into the patient 102 to enable blood to be drawn into the arterial line set 104 from the patient 102.

The venous line set 108 includes a venous line 128, a venous access line 130, and a venous drip chamber 132. The venous line 128 extends from the dialyzer 110 to a top of the venous drip chamber 132. One end of the venous access line 130 is connected to a bottom of the venous drip chamber 132, and the other end of the venous access line 130 is connected to a venous needle assembly 134. A pressure transducer 125b is connected to the venous drip chamber 132 via a pigtail line 131 extending from the venous drip chamber 132 and is configured to detect a fluid pressure within the venous drip chamber 132.

During an extracorporeal treatment, the venous needle assembly 134 is connected to the venous access line 130. The venous needle assembly 134 includes a needle 135 that is insertable into the patient 102 to enable filtered blood, e.g., blood that has travelled through the dialyzer 110, to be returned to the patient 102 through the venous line set 108. The venous needle assembly 134 further includes a fluid line 137 to be connected to the venous access line 130. A connector 141c at the end of the fluid line 137 is configured to be connected to a connector 141d at the end of the venous access line 130. The connectors 141c and 141d may be manually operable in some embodiments.

The arterial line set 104, and the venous line set 108 form an extracorporeal blood circuit through which the blood of the patient 102 circulates. The blood pump 118, when operated during the extracorporeal treatment, causes blood to flow from the patient 102, through the extracorporeal blood circuit and the dialyzer 110, and then back into the patient 102 after filtration has occurred in the dialyzer 110. The blood pump 118 generates a negative pressure in the arterial access line 120. A signal generated by the pressure transducer 125a can be indicative of this negative pressure. The blood pump 118 generates a positive pressure in the arterial line 122 and in the venous line set 108. For example, the blood pump 118 generates a negative pressure in a portion of the arterial line 122 upstream of the blood pump 118 and generates a positive pressure in a portion of the arterial line 122 downstream of the blood pump 118. A signal generated by the pressure transducer 125b can be indicative of the positive pressure in the downstream portion of the arterial line 122 and the venous line set 108.

When flow through the arterial line set 104 is inhibited, e.g., due to the operation of flow regulators (e.g., clamps) that interact with the arterial line set 104, the negative pressure generated in the arterial line set 104 can be maintained until the flow regulators are operated to allow blood to flow through the arterial line set 104 and the venous line set 108. When the flow regulators are operated to allow the blood to flow, as described herein, the negative pressure causes blood to be drawn further into the arterial line set 104 for reinfusion back to the patient 102.

Disposable portions of the extracorporeal system 100 include the fluid line 114 and the fluid source 112. The fluid line 114 is connected to the arterial line set 104 along the arterial line 122. For example, a port 139 of the arterial line set 104 extends from the fluid adapter 119 of the arterial line set 104 and is connectable to the fluid source 112, e.g., through the fluid line 114. An end of the port 139 includes a manually operable connector 141e configured to be connected to a manually operable connector 141f at an end of the fluid line 114. The connectors 141a-141f described herein can be threaded connectors that connect to one another through a threaded engagement, a snap-fit engagement, or another appropriate engagement mechanism.

The fluid line 114 is connected to the fluid source 112 such that fluid from the fluid source 112 can be drawn into the arterial line set 104 when the fluid line 114 is connected to the port 139 of the arterial line set 104. The fluid source 112 can include, for example, one or more of a priming solution, a substitution fluid, saline, or other medical fluids. For example, the fluid source 112 is a saline bag that can be hung from a vertically extending member 136 (shown in FIG. 1) that positions the fluid source 112 above the patient 102. The fluid source 112 is positioned above the patient 102 such that flow from the fluid source 112 to the patient 102, e.g., through the arterial line set 104 and the venous line set 10, can be driven by gravity.

The machine 106 further includes one or more flow regulators engageable with the arterial line set 104, the venous line set 108, and the fluid line 114. The flow regulators can be manually operable, electronically addressable, or both. In the embodiment illustrated in FIG. 2, for example, the machine 106 includes clamps 138a-138g, the clamps 138a-138f being manually operable and the clamp 138d being automated. The clamp 138 a is positioned to engage the fluid line 129 of the arterial needle assembly 126. The clamp 138b is positioned to engage the arterial access line 120. The clamp 138c is positioned to engage the port 139 of the arterial line set 104. The clamp 138d is positioned to engage the portion of the venous access line 130 proximate the venous drip chamber 132. The clamp 138e is positioned to engage the portion of the venous access line 130 proximate the venous needle assembly 134. The clamp 138f is positioned to engage the fluid line 137 of the venous needle assembly 134. The clamp 138g is positioned to engage the fluid line 114. The clamps 138a-138g can be independently actuated to control fluid flows through the arterial line set 104, the venous line set 108, and the fluid line 114.

In some implementations, the machine 106 includes one or more fluid flow sensors. For example, a fluid flow sensor 140a can be positioned to detect fluid flow through the arterial drip chamber 124, and a fluid flow sensor 140b can be positioned to detect fluid flow through the venous drip chamber 132. The fluid flow sensors 140a, 140b can be optical sensors responsive to drops of fluid through the arterial drip chamber 124 and the venous drip chamber 132, respectively. The fluid flow sensors 140a, 140b can detect flow rates of fluid flowing through the arterial drip chamber 124 and the venous drip chamber 132, respectively. In addition, the fluid flow sensors 140a, 140b can distinguish between fluids having different opacities, such as blood and saline. For example, during the operation of the blood pump 118, the type of fluid flowing through the arterial drip chamber 124 and the venous drip chamber 132 may vary depending on the stage of the extracorporeal treatment or the blood reinfusion process. The fluid flow sensors 140a, 140b can distinguish between the several types of fluid and provide the controller 144 with a signal indicative of a current stage of the extracorporeal treatment or the blood reinfusion process.

Whereas the arterial line set 104, the dialyzer 110, and the venous line set 108 form the extracorporeal blood circuit, the machine 106 includes components that form a dialysis fluid circuit with the dialyzer 110. These components include dialysate lines located inside the machine 106 and thus are not visible in FIG. 1. The extracorporeal blood circuit and the dialysis fluid circuit extend alongside one another through the dialyzer 110 such that the blood and the dialysis fluid flow adjacent to one another through the dialyzer 110 during an extracorporeal treatment. Flow of the blood and the dialysis fluid through the dialyzer 110 filters the blood by causing waste substances from the blood to diffuse into the dialysis fluid.

In some implementations, the machine 106 also includes an ultrafiltration pump 142 (shown in FIG. 1) to draw liquid, e.g., water, from the blood circulating through the dialyzer 110. The ultrafiltration pump 142 generates a pressure on the dialysis fluid circuit, thereby creating a pressure differential between the dialysis fluid circuit and the extracorporeal blood circuit. This pressure differential can cause liquid, e.g., water, to be withdrawn from the blood of the patient 102 through the dialyzer 110. During the ultrafiltration process, water is drawn from the extracorporeal blood circuit through the dialyzer, and into a waste line of the dialysis fluid circuit.

The machine 106 also includes a dialysis fluid pump 143 (shown in FIG. 1) operably connected to the dialysis fluid circuit. During an extracorporeal treatment, the dialysis fluid pump 143 is operated to circulate the dialysis fluid through the dialysis fluid circuit. The dialysis fluid pump 143 draws dialysis fluid from a dialysis fluid source, through the dialyzer, and into a dialysis fluid drain.

The machine 106 further includes a controller 144 (shown in FIG. 1) operably connected to the blood pump 118, the pressure transducers 125a, 125b, the fluid flow sensors 140a, 140b, the ultrafiltration pump 142, and the dialysis fluid pump 143. The controller 144 automatically controls the operations of the blood pump 118 during the extracorporeal treatment and the blood reinfusion process. As described herein, in some implementations, the controller 144 operates the blood pump 118 based on flow rates detected by one or more of the fluid flow sensors 140a, 140b. Alternatively, or additionally, the controller 144 operates the blood pump 118 based on pressures detected by one or more of the pressure transducers 125a, 125b.

In some implementations, a user interface system 150 is operable by the operator to monitor and control the operations of the machine 106. The user interface system 150 includes a touchscreen 151 and a display 152. The operator can manually operate the touchscreen 151 to control the functions of the machine 106, and the display 152 can provide visual indications to the operator. The user interface system 150 is integral to the machine 106 in some embodiments.

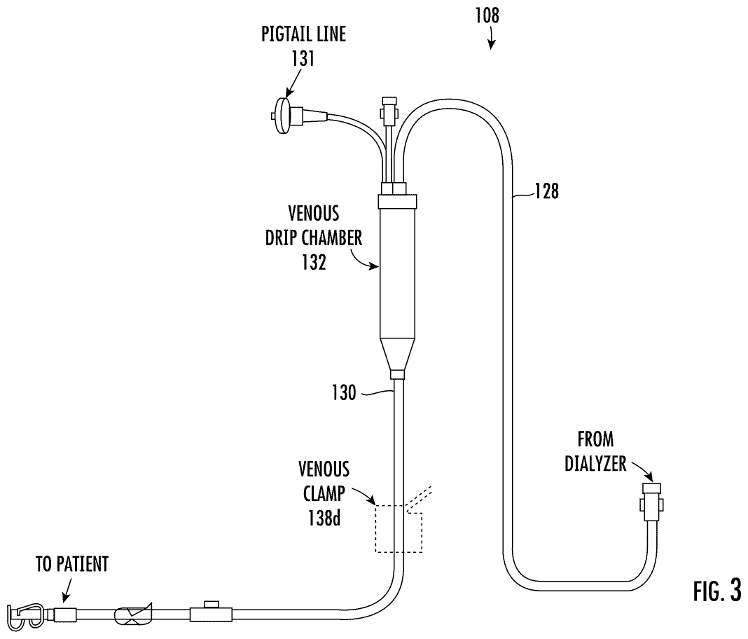

FIG. 3 depicts a portion of the extracorporeal circuit illustrating the portion of the venous line set 108 in which the venous drip chamber 132 operates. As described herein, the venous line set 108 includes a venous line 128, a venous access line 130, and a venous drip chamber 132. The venous line 128 extends from the dialyzer 110 to the venous drip chamber 132, and the venous access line 130 is connected to the venous drip chamber 132 at one end, and a venous needle assembly 134 to the patient 102 at another end (see FIG. 2). A pigtail line 131 extends from the venous drip chamber 132 and is configured to detect a fluid pressure within the venous drip chamber 132 using one or more sensors. A venous clamp 138d regulates the flow of fluid through the venous access line 130. The venous clamp 138d can be automatically or manually operated. The venous line set 108, using the venous drip chamber 132, facilitates that blood is gently returned to the patient 102 without sudden pressure or pump speed changes affecting the patient's fistula. However, this requires air in the venous drip chamber 132 for a separation between the force of the arterial pump and the venous portion of the extracorporeal circuit. However, air can potentially enter the extracorporeal circuit at various points like tubing connections or where the pressure is monitored. The air causes clotting over time, so conventional techniques typically use anti-clotting measures, such as anti-clotting compounds like heparin infused in the bloodline or Citrasate in the dialysate, to minimize clotting. However, neither of these options works for every patient 102. Furthermore, such compounds are expensive.

Accordingly, limiting exposure of blood in the venous line set 108 to air while also allowing air in the venous drip chamber 132 is a technical challenge. Embodiments described herein facilitate a technical solution to such technical challenges by using a bottom entry/bottom exit (BEBE) venous drip chamber with a filter.

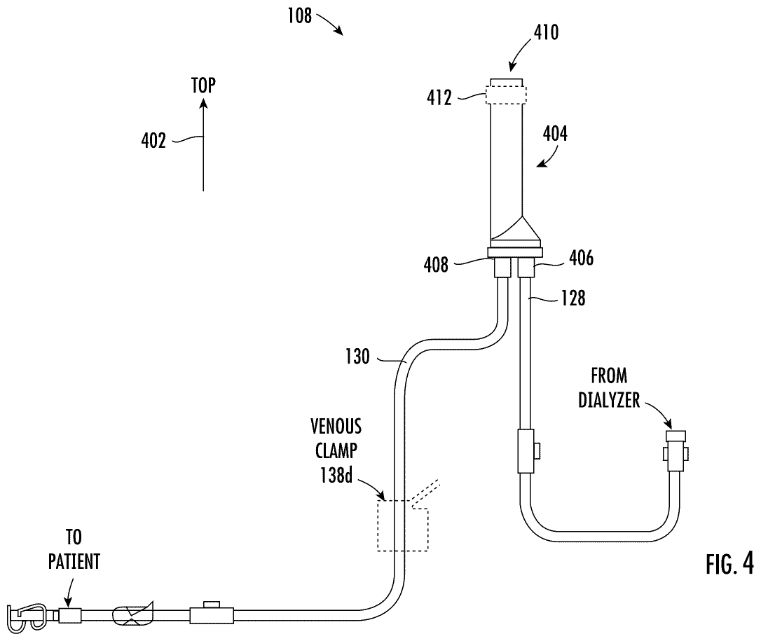

FIG. 4 depicts a venous line set using a BEBE drip chamber according to one or more embodiments. The top-vented BEBE drip chamber 404 provides a technical solution to limit the exposure of blood in the venous line set 108 to air. It is understood that the shape and dimensions of the BEBE drip chamber 404 can be different from the illustration unless explicitly stated otherwise herein. The directional terms, such as “top” and “bottom,” are used herein in reference to a direction map 402. As such, it is understood that other terms can be used to describe the positions/directions associated with the embodiments herein and that the different terms do not alter the effects of the features provided by the embodiments described herein.

The BEBE drip chamber 404 includes an entry port 406 and an exit port 408, both at the bottom end of the BEBE drip chamber 404. The venous line 128 from the dialyzer connects to the entry port 406, and the venous access line 130 connects to the exit port 408 and is directed to the patient 102.

Further, the BEBE drip chamber 404 includes a vent 410 at the top end. The vent 410 may be a self-sealing vent in some embodiments. In some embodiments, the vent 410 is a condensation-free vent. In some embodiments, the vent 410 is a solid disc made out of a blend of Polyethylene and Carboxymethylcellulose. The vent may have pores of pore size 15-45 microns, allowing air to pass. If a liquid (e.g., blood) comes into contact with the disc, the Carboxymethylcellulose expands and closes off the pores.

The BEBE drip chamber 404 further includes a membrane 412 proximate to the vent 410. The membrane 412 may be at a predetermined distance from the vent 410. In some embodiments, the vent 410 includes the membrane 412. The membrane 412 may be porous with pores of predetermined dimensions (1 nanometer, 1.5 nanometers, etc.) arranged in a predetermined pattern (e.g., circular, hexagonal, etc.). The membrane 412 may be made of microporous materials, such as zeolites and metal-organic frameworks. Alternatively, or in addition, the membrane 412 may be made from polymers, Polytetrafluoroethylene (PTFE), polyurethane (PU), polyolefins, polyamides, polyester, polyether, polyether-based copolymers, etc.

The membrane 412 facilitates avoiding the problem of condensation, e.g., on the filter, by directly venting the high-humidity air to the atmosphere through the micro-porous membrane 412 and the self-sealing vent 410 with no air space between the two. To prevent blood from leaving the BEBE drip chamber 404 during a blood leak through the micro-porous membrane 412, the self-sealing vent 410 expands and seals if it comes into contact with blood (or any other specific fluid).

In some embodiments, prior to a treatment (e.g., hemodialysis) using the extracorporeal circuit, air is first removed from the venous line set 108 by priming the venous line set 108 and pushing air out through the vent 410 at the top of the BEBE drip chamber 404. During treatment, the possibility still exists for air to get into the venous line set 108. Should air be present in the blood, the blood with air bubbles flows in through the bottom of the BEBE drip chamber 404. The upper motion of the blood is impeded by gravity, and the blood becomes stagnant while the air continues to the top, towards the vent 410 of the BEBE drip chamber 404. The air is vented to the atmosphere through the membrane 412 at the vent 410. In some embodiments, the vent 410 is a self-sealing vent.

However, because of the several constraints necessary to make the extracorporeal circuit airless, a technical challenge still exists because the BEBE chamber cannot reliably prevent fluid from wetting the vent 410. As described, once the vent 410 comes in contact with blood or any other fluid, the treatment has to be stopped or restarted. Thus, a more reliable solution is needed to limit blood exposure in the venous line set 108 to air and yet allow air in the venous drip chamber 132. Embodiments described herein facilitate further improvements and solutions to address such technical challenges by creating an ultrasonic barrier at the top of the BEBE drip chamber 404. The ultrasonic barrier uses signals at frequencies such that larger molecules and particles of fluids, like water, plasma, red blood cells, etc. are continually pushed away from the vent 410. In comparison, smaller molecules of gases, like those in air (O₂, N₂, Ar, CO₂, etc.), can pass by unimpeded through the vent 410.

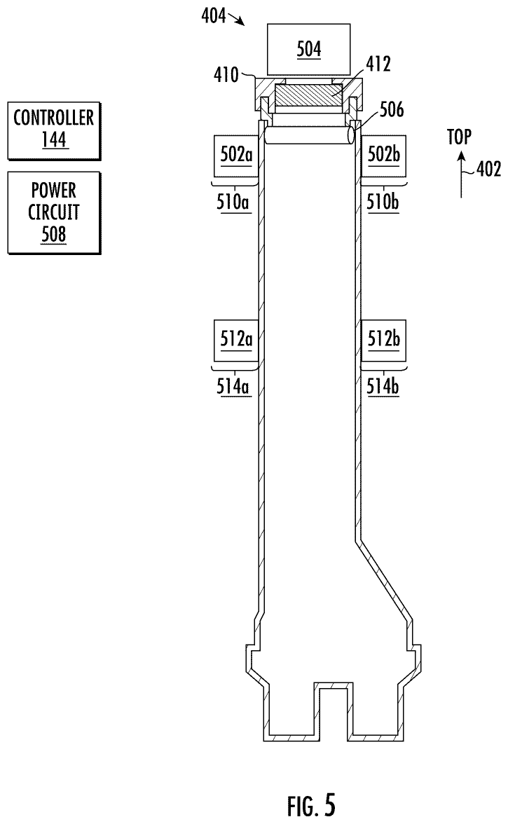

FIG. 5 depicts a BEBE drip chamber 404 with an ultrasonic barrier 506 according to one or more embodiments. The depicted BEBE drip chamber 404 is one example, and the dimensions, shape, and other such parameters of the BEBE drip chamber 404 can vary in other embodiments unless explicitly stated otherwise.

The BEBE drip chamber 404 in FIG. 5 includes an ultrasonic barrier 506 at the top of the BEBE drip chamber 404. The ultrasonic barrier 506 is created by a first array of ultrasonic transducers, including one or more ultrasonic transducers 502. It should be noted that an “array” of components as used herein can include one or more components. Ultrasonic transducers 502 are devices that generate and/or sense ultrasonic signals or ultrasound energy. The ultrasonic transducers convert electrical signals into ultrasonic signals, which create the ultrasonic barrier 506. In some embodiments, the ultrasonic transducers 502 are coupled with a power circuit 508. The power circuit 508 provides electric signals to the ultrasonic transducers 502. In some embodiments, each ultrasonic transducer 502 includes its separate power circuit 508. The power circuit 508 can include a power source, such as a battery. Alternatively, or in addition, the power source can include a circuit that converts electric signals from another power supply (e.g., electrical socket) to the specifications of the ultrasonic transducers 502. The power circuit 508 can include additional components like wires, chips, etc.

The ultrasonic transducers 502 (or “ultrasonic heads”) are positioned at a predetermined vicinity of the top of the BEBE drip chamber 404. The predetermined vicinity can be specified as a particular distance (e.g., 1 millimeter, 5 millimeters, 5 nanometers, etc.) from the top of the BEBE drip chamber 404, from a bottom of the vent 410, from the membrane 412, or in relation to any other specific position. The ultrasonic transducers 502 can be integrated into holders for the ultrasonic transducers 502 to line up with the desired barrier location. Each ultrasonic transducer 502a, 502b may have respective holders 510a, 510b. The holders 508 may be made from polyethylene plastic, thermoplastic polymer (e.g., Acrylonitrile butadiene styrene (ABS)), or any other such moldable material.

In some embodiments, the ultrasonic transducers 502 can generate ultrasonic signals of a predetermined frequency in the ultrasound spectrum (typically between about 20 kilohertz (kHz) to about 100 kHz). In various embodiments, the ultrasonic transducers 502 can generate ultrasonic signals of a predetermined frequency of about 5 kHz, about 10 kHz, about 20 kHz, about 30 kHz, about 40 kHz, about 50 kHz, about 60 kHz, about 70 kHz, about 80 kHz, about 90 kHz, about 100 kHz, about 125 kHz, about 150 kHz, about 200 kHz, greater than 200 kHz, and any value or range between any two of these values (including endpoints).

The predetermined frequency can be configured by supplying an appropriate amount of electric power from the power circuit 508. The power circuit 508 can be configured using the controller 144 in some embodiments. In other embodiments, a separate controller, for example, part of the power circuit 508 (not shown), may be used to configure the power circuit 508. The predetermined frequency can be selected to create the ultrasonic barrier 506 that affects molecules of at least a predetermined size (and conversely, does not affect molecules smaller than the predetermined size). For example, the predetermined frequency is selected such that the larger molecules, like water, plasma, red blood cells, etc., are filtered from the smaller molecules, like O₂, N₂, Ar, CO₂, etc. In other words, the predetermined frequency prevents the larger molecules from reaching the membrane 412 and allows the smaller molecules to reach the membrane 412. Accordingly, the smaller molecules, primarily the gases, can escape from the vent 410, whereas the larger molecules do not reach the membrane 412, preventing the membrane 412 from getting wet.

In some embodiments, the BEBE drip chamber 404 is equipped with a sensor array of one or more sensors 504. In some embodiments, the sensors 504 can be a second array of one or more ultrasonic transducers. The sensors 504 generate ultrasonic signals at a different predetermined frequency from the ultrasonic transducers 502. The sensors 504 can generate ultrasonic signals of a predetermined frequency in the ultrasound spectrum (typically between about 20 kilohertz (kHz) to about 100 kHz). In various embodiments, the sensors 504 can generate ultrasonic signals of a predetermined frequency of about 5 kHz, about 10 kHz, about 20 kHz, about 30 kHz, about 40 kHz, about 50 kHz, about 60 kHz, about 70 kHz, about 80 kHz, about 90 kHz, about 100 kHz, about 125 kHz, about 150 kHz, about 200 kHz, greater than 200 kHz, and any value or range between any two of these values (including endpoints).

Further, the sensors 504 are positioned differently, to be pointed down toward the top of the vent 410 to monitor the pore size of the vent 410 continually. The sensors 504 transmit and receive the ultrasonic signals of the second predetermined frequency, which are reflected from the BEBE drip chamber 404. Based on the received reflections of the ultrasonic signals, the sensors 504 detect if the pore size of the vent 410 has changed (e.g., reduced). If and when the sensors 504 detect the pores are closing due to liquid intrusion, the sensors 504 may trigger one or more intrusion events. Non-limiting examples of the intrusion events may include stopping the blood pump 118, closing the venous clamp 138d, alerting the user, and the like or a combination thereof. Here, the user can be the patient 102 and/or an operator of the extracorporeal circuit.

In other embodiments, the sensors 504 can use imaging, light, radio waves, or any other type of signals (other than ultrasonic signals), or a combination thereof, to detect the change in pore size of the vent 410. The sensors 504 can be configured using the controller 144 and/or any other controller (not shown) that may be coupled with the sensors 504. Further, the sensors 504 may be equipped with a separate power supply, such as a battery. Alternatively, or in addition, the sensors 504 may be coupled with the power circuit 508 to receive the requisite amount of electrical power to operate.

In some embodiments, the BEBE drip chamber 404 is equipped with another (third) array of ultrasonic transducers 512. The ultrasonic transducers 512 are positioned differently from the first array of ultrasonic transducers 502. The ultrasonic transducers 512 are positioned using a separate set of holders 514 (514a, 514b, etc.). The ultrasonic transducers 512 may be closer to the bottom of the BEBE drip chamber 404 compared to the ultrasonic transducers 502. In some embodiments, the ultrasonic transducers 512 generate ultrasonic signals at a different (third) predetermined frequency compared to the first predetermined frequency of the first array of ultrasonic transducers 502.

Additionally, the third predetermined frequency differs from the second predetermined frequency, which may be associated with the sensors 504. The third predetermined frequency is configured to actively prevent blood clots' formation in the BEBE drip chamber 404 fluid. For example, the ultrasonic transducers 512 emit continuous ultrasonic signals in low frequency in the range of approximately 20 Hertz that “stir” components of the blood, such as red blood cells, such that they cannot form clots. In another embodiment, the ultrasonic transducers 512 emit bursts of ultrasonic signals at approximately 50 Mega Hertz to sever long chains of red blood cells. In some embodiments, the ultrasonic transducers 512 can generate ultrasonic signals of a predetermined frequency in the ultrasound spectrum (typically between about 20 kilohertz (kHz) to about 100 kHz). In various embodiments, the ultrasonic transducers 512 can generate ultrasonic signals of a predetermined frequency of about 5 kHz, about 10 kHz, about 20 kHz, about 30 kHz, about 40 kHz, about 50 kHz, about 60 kHz, about 70 kHz, about 80 kHz, about 90 kHz, about 100 kHz, about 125 kHz, about 150 kHz, about 200 kHz, greater than 200 kHz, and any value or range between any two of these values (including endpoints).

Using the one or more arrays of sensors and ultrasonic transducers, the BEBE drip chamber 404 continually vents air in the extracorporeal circuit and monitors for failures and protects against clots.

It is understood that although only one or two elements are shown per array in FIG. 5, each array can include any integer number of elements in one or more embodiments. For example, the first array of ultrasonic transducers 502 can include two ultrasonic transducers; the second array of sensors 504 can include one sensor; and the third array of ultrasonic transducers 512 can include two ultrasonic transducers. In another example, the first array includes two ultrasonic transducers, the second array includes two sensors, and the third array includes four ultrasonic transducers. Any other combination is possible.

It should be understood that in some embodiments the different ultrasonic transducer arrays may have overlapping frequency ranges. Alternatively, the different ultrasonic transducer arrays may have distinct frequency ranges.

In some embodiments, the ultrasonic transducers (502, 512), sensors 504, and power circuit 508 are part of a separate device plugged into the extracorporeal circuit. In some embodiments, the device has a separate controller that is coupled for communication with the controller 144 of the extracorporeal circuit, either wired or wireless. Alternatively, the controller 144 can communicate with the transducers and sensors of the device directly through wired or wireless communication.

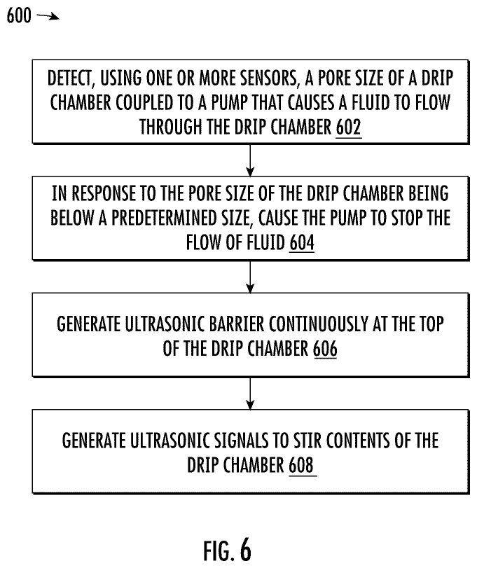

FIG. 6 illustrates an example method 600 according to one or more embodiments. The method includes operations for limiting exposure of the contents of the venous drip chamber 132 to air to prevent clots in the contents. Additionally, the method 600 as depicted includes operations for detecting pore size of the venous drip chamber and operations based on the detected pore size. Additionally, the method 600 includes operations to stir contents of the venous drip chamber. Although the example method 600 depicts a particular sequence of operations, the sequence may be altered without departing from the scope of the present disclosure. For example, some of the operations depicted may be performed in parallel or in a different sequence that does not materially affect the function of the method 600. In other examples, different components of an example device or system that implements the method 600 may perform functions simultaneously or in a specific sequence. In some examples, one or more operations depicted may not be performed.

According to some examples, the method includes detecting, using one or more sensors, a pore size of the venous drip chamber 132, which is coupled to the blood pump 118 that causes a fluid (e.g., blood) to flow through the venous drip chamber 132 at block 602. The one or more sensors can be the sensors 504. In some embodiments, the sensors 504 use ultrasonic signals to detect the pore size.

According to some examples, the method includes, in response to the pore size of the venous drip chamber 132 being below a predetermined size, causing the blood pump 118 to stop the flow of fluid at block 604. The flow may be stopped by signaling the venous clamp 138d to prevent the flow of fluid. Alternatively, or in addition, the flow may be stopped by signaling the blood pump 118 to stop or pause its operation. Such signaling may be performed by the controller 144. The controller 144 sends such signals to the blood pump 118 and/or the venous clamp 138d based on detecting and comparing the pore size with the predetermined size. The controller 144 may perform the comparison in some embodiments. The controller 144 performs such a comparison based on the sensors 504 providing the detected pore size to the controller 144. In other embodiments, the controller 144 may receive an indication of the comparison and, based on the indication, may signal the blood pump 118 and/or the venous clamp 138d.

In other embodiments, the controller 144 may notify a user, such as the patient 102 and/or an operator, to cause the extracorporeal circuit to stop operating based on the detected pore size.

Further, the method 600 includes generating the ultrasonic barrier 506 at the top of the venous drip chamber 132 at block 606. The controller 144 may cause the (first array) of ultrasonic transducers 502 (502a, 502b) to generate the ultrasonic signals that result in the ultrasonic barrier 506. The controller 144 may cause the ultrasonic barrier 506 to be generated continuously in some embodiments based on, and as long as the blood pump 118 is operational. The ultrasonic barrier 506 is generated according to the first predetermined frequency of ultrasonic signals selected.

The method 600 further includes generating ultrasonic signals to stir the venous drip chamber 132 contents at block 608. The controller 144 may cause the (third array) of ultrasonic transducers 512 (512a, 512b) to generate the ultrasonic signals that result in stirring contents of the venous drip chamber 132. The controller 144 may cause the ultrasonic signals to be generated continuously in some embodiments based on, and as long as the blood pump 118 is operational. In some embodiments, the controller 144 may cause the ultrasonic signals to be generated at predetermined intervals (e.g., every 2 seconds, every 5 seconds, etc.). Alternatively, or in addition, the controller 144 may cause the ultrasonic signals to be generated by the ultrasonic transducers 512 based on the detected pore size. For example, suppose the pore size is determined to be smaller than the predetermined size. In that case, the controller 144 may cause the ultrasonic transducers 512 to generate the ultrasonic signals to stir the contents to potentially reduce clots in the contents. The ultrasonic signals are generated according to the third predetermined frequency of ultrasonic signals selected.

Embodiments described herein provide a device for monitoring an extracorporeal circuit in which a blood pump is disposed to convey blood. The device includes one or more ultrasonic transducers positioned in a predetermined vicinity of a vent of a drip chamber of the extracorporeal circuit. The device further includes a controller coupled with the one or more ultrasonic transducers. The controller causes the one or more ultrasonic transducers to emit ultrasonic signals that displace particles in the contents of the drip chamber away from the vent.

In some embodiments, the controller causes the one or more ultrasonic transducers to emit the ultrasonic signals of a predetermined frequency, the ultrasonic signals of the predetermined frequency only displace the particles that are larger than a predetermined size. The particles that are larger than the predetermined size cause condensation on the vent.

In some embodiments, the particles that are displaced by the one or more ultrasonic transducers comprise molecules of water, plasma, and red blood cells.

In some embodiments, the ultrasonic signals of the predetermined frequency allow the particles that are smaller than the predetermined size to pass through the vent.

In some embodiments, the device further includes one or more sensors to monitor a pore size of the drip chamber.

In some embodiments, the one or more sensors include a second set of ultrasonic transducers, wherein the controller is coupled with the second set of ultrasonic transducers to cause the second set of ultrasonic transducers to emit ultrasonic signals of a second predetermined frequency.

In some embodiments, in response to the pore size being detected to be less than a predetermined threshold, the controller performs an operation, such as stopping the blood pump, closing a venous clamp, and alerting a user.

In some embodiments, the one or more ultrasonic transducers is a first set of ultrasonic transducers, the predetermined frequency is a first predetermined frequency, and the device further includes a second set of ultrasonic transducers that are coupled with the controller. The controller causes the second set of ultrasonic transducers to emit ultrasonic signals of a second predetermined frequency, different from the first predetermined frequency, the ultrasonic signals of the second predetermined frequency prevent blood clots in the contents of the drip chamber.

In some embodiments, the second set of ultrasonic transducers emits ultrasonic signals continuously that prevent red blood cells from clotting.

In some embodiments, the second set of ultrasonic transducers emits ultrasonic signals of a third predetermined frequency, different from the first and second predetermined frequencies, the ultrasonic signals of the third predetermined frequency sever chains of red blood cells. The third predetermined frequency of ultrasonic signals is emitted only at predetermined intervals.

In some embodiments, the device is part of a dialysis system, such as a hemodialysis system. In other embodiments, the device is plugged into a dialysis system. In yet other embodiments, the device may be used with other types of systems, such as fluid filtration (e.g., oil refining), fluid injection (e.g., fuel injection), fluid mixing (e.g., food products machinery), etc.

Some embodiments of the disclosed system may be implemented, for example, using a storage medium, a computer-readable medium, or an article of manufacture which may store an instruction or a set of instructions that, if executed by a machine (i.e., processor or microcontroller), may cause the machine to perform a method and/or operations in accordance with embodiments of the disclosure. In addition, a server or database server may include machine-readable media configured to store machine-executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. In addition, while certain embodiments have been described and illustrated with certain features, it is envisioned that features of one embodiment may be used in combination with other embodiments. Moreover, the following claims are hereby incorporated into this Detailed Description by this reference, with each claim standing on its own as a separate embodiment of the present disclosure.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A device for monitoring an extracorporeal circuit in which a blood pump is disposed to convey blood, the device comprising:
one or more ultrasonic transducers positioned in a predetermined vicinity of a vent of a drip chamber of the extracorporeal circuit; and
a controller coupled to the one or more ultrasonic transducers, the controller configured to cause the one or more ultrasonic transducers to emit ultrasonic signals that displace particles in contents of the drip chamber away from the vent.

2. The device of claim 1, wherein the controller causes the one or more ultrasonic transducers to emit the ultrasonic signals of a predetermined frequency, the predetermined frequency configured to displace the particles larger than a predetermined size.

3. The device of claim 2, wherein the particles that are larger than the predetermined size cause condensation on the vent.

4. The device of claim 2, wherein the particles that are displaced by the one or more ultrasonic transducers comprise one or more of water, plasma, and red blood cells.

5. The device of claim 2, wherein the ultrasonic signals of the predetermined frequency allow the particles that are smaller than the predetermined size to pass through the vent.

6. The device of claim 1, further comprising one or more sensors to monitor a pore size of the drip chamber.

7. The device of claim 6, wherein the one or more sensors comprise a second set of ultrasonic transducers, wherein the controller is coupled with the second set of ultrasonic transducers, the controller configured to cause the second set of ultrasonic transducers to emit ultrasonic signals of a second predetermined frequency.

8. The device of claim 6, wherein, in response to the pore size being detected to be less than a predetermined threshold, the controller performs at least one from a group of operations, the group of operations comprises, stopping the blood pump, closing a venous clamp, and alerting a user.

9. The device of claim 2, wherein the one or more ultrasonic transducers is a first set of ultrasonic transducers, the predetermined frequency is a first predetermined frequency, and the device comprises a second set of ultrasonic transducers that are coupled with the controller, which causes the second set of ultrasonic transducers to emit ultrasonic signals of a second predetermined frequency, different from the first predetermined frequency, the ultrasonic signals of the second predetermined frequency prevent blood clots in the contents of the drip chamber.

10. The device of claim 9, wherein the second set of ultrasonic transducers emits ultrasonic signals continuously that prevents red blood cells from clotting.

11. The device of claim 9, wherein the second set of ultrasonic transducers emits ultrasonic signals of a third predetermined frequency, different from the first and second predetermined frequencies, the ultrasonic signals of the third predetermined frequency sever chains of red blood cells.

12. A dialysis system, comprising:
a venous drip chamber comprising a vent;
one or more ultrasonic transducers positioned in a predetermined vicinity of the vent; and
a controller coupled with the one or more ultrasonic transducers, the controller configured to cause the one or more ultrasonic transducers to emit ultrasonic signals that displace particles in contents of the venous drip chamber away from the vent.

13. The dialysis system of claim 12, wherein the venous drip chamber is top-vented.

14. The dialysis system of claim 12, wherein the controller causes the one or more ultrasonic transducers to emit the ultrasonic signals of a predetermined frequency, the predetermined frequency selected to displace the particles larger than a predetermined size.

15. The dialysis system of claim 14, further comprising one or more sensors to monitor a pore size of the drip chamber.

16. The dialysis system of claim 15, wherein, in response to the pore size being detected to be less than a predetermined threshold, the controller performs at least one from a group of operations, the group of operations comprises, stopping a blood pump, closing a venous clamp, and alerting a user.

17. The dialysis system of claim 16, wherein the one or more sensors comprise a second set of ultrasonic transducers, wherein the controller is coupled with the second set of ultrasonic transducers, the controller configured to cause the second set of ultrasonic transducers to emit ultrasonic signals of a second predetermined frequency.

18. The dialysis system of claim 14, wherein the one or more ultrasonic transducers is a first set of ultrasonic transducers, the predetermined frequency is a first predetermined frequency, and the dialysis system comprises a second set of ultrasonic transducers that are coupled with the controller, which causes the second set of ultrasonic transducers to emit ultrasonic signals of a second predetermined frequency, different from the first predetermined frequency, the ultrasonic signals of the second predetermined frequency prevent blood clots in the contents of the venous drip chamber.

19. The dialysis system of claim 18, wherein the controller causes the second set of ultrasonic transducers to perform at least one of:
emit ultrasonic signals continuously to prevent red blood cells from clotting; and
emit ultrasonic signals of a third predetermined frequency, different from the first and second predetermined frequencies, the ultrasonic signals of the third predetermined frequency sever chains of red blood cells.

20. A computer-implemented method, comprising:
detecting, using one or more sensors, a pore size of a drip chamber coupled to a pump that causes a fluid to flow through the drip chamber; and
in response to the pore size of the drip chamber being below a predetermined size, causing the pump to stop the flow of fluid.
